(19) United States
(12) Patent Application Publication
(10) Pub. No.: US 2009/0215133 A1
(43) Pub. Date: Aug. 27, 2009

(54) PRODUCTION OF L-LYSINE AND L-LYSINE-CONTAINING FEED ADDITIVES

(75) Inventors: BRIGITTE BATHE, SALZKOTTEN (DE); VOLKER F. WENDISCH, JULICH (DE)

Correspondence Address: PILLSBURY WINTHROP SHAW PITTMAN, LLP, P.O. BOX 10500, MCLEAN, VA, 22102, US

(73) Assignee: EVONIK DEGUSSA GMBH, ESSEN (DE)

(21) Appl. No.: 12/024,587

(22) Filed: Feb. 1, 2008

Related U.S. Application Data
(60) Provisional application No. 60/887,902, filed on Feb. 2, 2007.
(60) Provisional application No. 60/989,722, filed on Nov. 21, 2007.

(51) Int. Cl. C12P 13/08; C12N 1/21; A23K 1/16

(52) U.S. Cl. 435/115; 435/252.3; 435/252.32; 435/252.33; 426/7

(57) ABSTRACT

The invention relates to a process for producing L-lysine or L-lysine-containing feed additives by fermentation, which comprises
a) expressing a polynucleotide coding for polypeptide having LL-diaminopimelate aminotransferase activity in a bacterium excreting L-lysine, and
b) fermenting the resultant bacterium in a medium under suitable conditions and allowing the resultant L-lysine to accumulate in the fermentation broth.

The invention relates to a process for producing L-lysine or L-lysine-containing feed additives using bacteria in which a polynucleotide coding for a polypeptide having LL-diaminopimelate aminotransferase activity is expressed.

### PRIOR ART

L-Lysine and its salts are used in human medicine, in the pharmaceutical industry, in the food industry and very particularly in animal nutrition.

It is known that L-lysine is produced by fermentation of strains of coryneform bacteria, in particular Corynebacterium glutamicum, and Enterobacteriaceae, in particular Escherichia coli. Because of its great importance, work is continuously proceeding on improving the production process. Process improvements can relate to fermentation measures such as, for example, agitation and supply with oxygen, or composition of the nutrient media, such as, for example, the sugar concentration during fermentation, or workup to give the product form by, for example, ion-exchange chromatography or the intrinsic performance properties of the bacterium itself.

To improve the performance properties of these microorganisms, use is made of methods of mutagenesis, selection and mutant selection. In this manner, strains are obtained which are resistant to antimetabolites or are auxotrophic for metabolites of regulatory importance and which produce amino acids. A known antimetabolite is the lysine analog S-(2-aminoethyl)-L-cysteine (AEC).

For some years, likewise use has been made of methods of recombinant DNA technology for strain improvement of L-lysine-producing strains by amplifying individual amino acid biosynthesis genes and studying the effect on amino acid production.

The chromosome of Escherichia coli was completely sequenced sometime ago (Blattner et al., Science 277, 1453-1462 (1997)).

The chromosome of Corynebacterium glutamicum has recently likewise been completely sequenced (Kalinowski et al., Journal of Biotechnology 104, 5-25 (2003)). The chromosome of Corynebacterium efficiens has likewise already been sequenced (Nishio et al., Genome Res. 13 (7), 1572-1579 (2003)).

Corresponding sequence data can be taken from the public databases. Suitable databases are, for example, the database of the European Molecular Biologies Laboratories (EMBL, Heidelberg, Germany and Cambridge, UK), the database of the National Center for Biotechnology Information (NCBI, Bethesda, Md., USA), that of the Swiss Institute of Bioinformatics (Swissprot, Geneva, Switzerland), the Protein Information Resource Database (PIR, Washington, D.C., USA) and the DNA Data Bank of Japan (DDBJ, 1111 Yata, Mishima, 411-8540, Japan).

Summarizing information on the cell and molecular biology of Escherichia coli may be found in the textbook by Neidhardt (ed): Escherichia coli and Salmonella, Cellular and Molecular Biology, 2nd edition, ASM Press, Washington, D.C., USA, (1996).

Summarizing accounts on the genetics, metabolism and technical importance of Corynebacterium may be found in the papers by Ikeda, Pfefferle et al. and Mueller and Huebner in the book “Microbial Production of L-Amino Acids” (Advances in Biochemical Engineering 79, (2003), Springer Verlag, Berlin, Germany, editor: T. Scheper), in the special edition “A New Era in Corynebacterium glutamicum Biotechnology” of the Journal of Biotechnology (volume 104 (1-3), 2003, editors: A. Puhler and T. Tauch) and in the “Handbook of Corynebacterium glutamicum” (editors: L. Eggeling and M. Bott, CRC Press, Taylor & Francis Group, Boca Raton, Fla., USA, 2005).

In bacteria, three lysine biosynthesis pathways are known which are called the “succinylase pathway”, “acetylase pathway” and “dehydrogenase pathway” (Schrumpf et al., Journal of Bacteriology 173(14), 4510-4516 (1991)).

### OBJECT OF THE INVENTION

The object of the invention is to provide novel measures for improved production of L-lysine or L-lysine-containing feed additives.

### DESCRIPTION OF THE INVENTION

The invention relates to a process for producing L-lysine or L-lysine-containing feed additives by fermentation, which comprises
a) expressing a polynucleotide coding for a polypeptide having LL-diaminopimelate aminotransferase activity in a bacterium excreting L-lysine preferably selected from the group coryneform bacteria, Enterobacteriaceae and Bacillus, and
b) fermenting the resultant bacterium in a medium at a desired temperature and allowing the resultant L-lysine to accumulate in the fermentation broth.

Preference is given to polynucleotides, if appropriate in the form of the cDNA, which code for polypeptides having LL-diaminopimelate aminotransferase activity, from Arabidopsis thaliana, Synechococcus, Oryza sativa and Vitis vinifera.

Reports on the structure of LL-diaminopimelate aminotransferase (LL-DAP-AT) of Arabidopsis thaliana and on biochemical properties of the protein are described in Hudson et al. (Plant Physiology 140, 292-301 (2006)) and in WO 07/053,203.

For better clarity, the amino acid sequences of LL-diaminopimelate aminotransferase of Arabidopsis thaliana, Synechococcus and Oryza sativa are reproduced in SEQ ID NO: 2, SEQ ID NO: 4 and SEQ ID NO: 6. The nucleotide sequences of the coding regions corresponding to the cDNA are given in SEQ ID NO: 1, SEQ ID NO: 3 and SEQ ID NO: 5.

The amino acid sequence of the LL-diaminopimelate aminotransferase of Vitis vinifera is shown in SEQ ID NO: 10. The corresponding nucleotide sequence of the coding region is reproduced in SEQ ID NO: 9.

For the measures of the invention according to a), use is made of those strains of bacteria (starting strains or parent strains) which already possess the ability to enrich L-lysine in the cell and/or excrete it into the nutrient medium surrounding the cell or to accumulate it in the fermentation broth. The expression “produce” can also be used for this. In particular, the strains used for the measures of the invention possess the ability to enrich or accumulate in the cell and/or in the nutrient medium (at least) 0.25 g/l, 0.5 g/l, 1.0 g/l, 1.5 g/l, 2.0 g/l, 4 g/l or 10 g/l of L-lysine in (maximally) 120 hours, 96 hours, 48 hours, 36 hours, 24 hours or 12 hours. These can be strains which were produced by mutagenesis and selection, by recombinant DNA techniques or by a combination of both methods.

In the case of the coryneform bacteria, the genus Corynebacterium is preferred. In the case of the genus Corynebacterium, strains are preferred which are based on the following species:
Corynebacterium efficiens, such as, for example, the type strain DSM44549,
Corynebacterium glutamicum, such as, for example, the type strain ATCC13032, or the strain R, and
Corynebacterium ammoniagenes, such as, for example, the strain ATCC6871,
   the species Corynebacterium glutamicum being very particularly preferred.

Some representatives of the species Corynebacterium glutamicum are also known under other names in the prior art. These include, for example:
Corynebacterium acetoacidophilum ATCC13870,
Corynebacterium lilium DSM20137,
Corynebacterium melassecola ATCC17965,
Brevibacterium flavum ATCC14067,
Brevibacterium lactofermentum ATCC13869, and
Brevibacterium divaricatum ATCC14020.

The term “Micrococcus glutamicus” for Corynebacterium glutamicum was likewise customary.

Some representatives of the species Corynebacterium efficiens have also been referred to in the prior art as Corynebacterium thermoaminogenes, such as, for example, the strain FERM BP-1539.

Known representatives of L-lysine-excreting strains of the genus Corynebacterium are, for example:
Corynebacterium glutamicum DM58-1/pDM6 (=DSM4697) described in EP 0 358 940,
Corynebacterium glutamicum MH20-22B (=DSM16835) described in Menkel et al. (Applied and Environmental Microbiology 55(3), 684-688 (1989)),
Corynebacterium glutamicum AHP-3 (=Ferm BP-7382) described in EP 1 108 790,
Corynebacterium glutamicum DSM16834 described in (PCT/EP2005/012417),
Corynebacterium glutamicum DSM17119 described in (PCT/EP2006/060851),
Corynebacterium glutamicum DSM17223 described in (PCT/EP2006/062010),
Corynebacterium glutamicum DSM16937 described in (PCT/EP2005/057216), and
Corynebacterium thermoaminogenes AJ12521 (=FERM BP-3304) described in U.S. Pat. No. 5,250,423.

Details on the taxonomic classification of strains of this group of bacteria are found, inter alia, in Seiler (Journal of General Microbiology 129, 1433-1477 (1983)), Kinoshita (1985, Glutamic Acid Bacteria, p 115-142. In: Demain and Solomon (ed), Biology of Industrial Microorganisms. The Benjamin/Cummins Publishing Co., London, UK), Kämpfer and Kroppenstedt (Canadian Journal of Microbiology 42, 989-1005 (1996)), Liebl et al (International Journal of Systematic Bacteriology 41, 255-260 (1991)). Fudou et al (International Journal of Systematic and Evolutionary Microbiology 52, 1127-1131 (2002)) and in U.S. Pat. No. 5,250,434.

Strains with the designation “ATCC” are available from the American Type Culture Collection (Manassas, Va., USA). Strains with the designation “DSM” are available from the Deutsche Sammlung von Mikroorganismen und Zellkulturen (DSMZ, Brunswick, Germany). Strains with the designation “NRRL” are available from the Agricultural Research Service Patent Culture Collection (ARS, Peoria, Ill., US). Strains with the designation “FERM” are available from the National Institute of Advanced Industrial Science and Technology (AIST Tsukuba Central 6, 1-1-1 Higashi, Tsukuba Ibaraki, Japan).

L-Lysine-producing coryneform bacteria typically possess a feedback resistant or desensitized aspartate kinase. Feedback resistant aspartate kinases are taken to mean aspartate kinases (LysC) which, in comparison to the wild type, have a lower sensitivity to inhibition by mixtures of lysine and threonine, or mixtures of AEC (aminoethylcysteine) and threonine, or lysine alone, or AEC alone. The genes or alleles coding for these desensitized aspartate kinases are also termed lysC^{FBR} alleles. In the prior art numerous lysC^{FBR} alleles are described which code for aspartate kinase variants which have amino acid replacements compared with the wild type protein. The coding region of the wild type lysC gene of Corynebacterium glutamicum corresponding to the accession number AX756575 of the NCBI database is shown in SEQ ID NO: 7 and the polypeptide coded by this gene is shown in SEQ ID NO: 8. It has been established that the wild type aspartate kinase of strain ATCC 14067 at position 317 contains alanine. The wild type aspartate kinase of strain ATCC 13032 contains serine at this position, as shown in SEQ ID No: 8.

The L-lysine-producing coryneform bacteria used for the measures of the invention preferably have a lysC allele which codes for an aspartate kinase variant which possesses the amino acid sequence of SEQ ID NO: 8, with these comprising one or more of the amino acid replacements selected from the group:
LysC A279T (replacement of L-alanine at position 279 of the coded aspartate kinase protein according to SEQ ID NO: 8 by L-threonine; see U.S. Pat. No. 5,688,671 and accession numbers E06825, E06826, E08178 and 174588 to 174597),
LysC A279V (replacement of L-alanine at position 279 of the coded aspartate kinase protein according to SEQ ID NO: 8 by L-valine; see JP 6-261766 and accession number E08179),
LysC L297Q (replacement of L-leucine at position 297 of the coded aspartate kinase protein according to SEQ ID NO: 8 by L-glutamine; see DE 102006026328,
LysC S301F (replacement of L-serine at position 301 of the coded aspartate kinase protein according to SEQ ID NO: 12 by L-phenylalanine; see U.S. Pat. No. 6,844,176 and accession number E08180),
LysC S301Y (replacement of L-serine at position 301 of the coded aspartate kinase protein according to SEQ ID NO: 8 by L-tyrosine; see Kalinowski et al. (Molecular and General Genetics 224, 317-324 (1990)) and accession number X57226),
LysC T308I (replacement of L-threonine at position 308 of the coded aspartate kinase protein according to SEQ ID NO: 8 by L-isoleucine; see JP 6-261766 and accession number E08181),
LysC T311I (replacement of L-threonine at position 311 of the coded aspartate kinase protein according to SEQ ID NO: 8 by L-isoleucine; see WO 00/63388 and U.S. Pat. No. 6,893,848),
LysC R320G (replacement of L-arginine at position 320 of the coded aspartate kinase protein according to SEQ ID NO: 8 by glycine; see Jetten et al. (Applied Microbiology and Biotechnology 43, 76-82 (1995)) and accession number L27125),
LysC G345D (replacement of glycine at position 345 of the coded aspartate kinase protein according to SEQ ID NO: 8 by L-aspartic acid; see Jetten et al. (Applied Microbiology and Biotechnology 43, 76-82 (1995)) and accession number L16848),
LysC T380I (replacement of L-threonine at position 890 of the coded aspartate kinase protein according to SEQ ID NO: 8:10 by L-isoleucine; see WO 01/49854 and accession number AX192358), and
LysC S381F (replacement of L-serine at position 891 of the coded aspartate kinase protein according to SEQ ID NO: 8 by L-phenylalanine; see EP 0435132),
   where, if appropriate, L-alanine may be present at position 317 instead of L-serine.

Particular preference is given to the lysC^{FBR} allele lysC T311I (replacement of threonine at position 311 of the coded aspartate kinase protein according to SEQ ID NO: 8 by isoleucine) and a lysC^{FBR} allele containing at least one replacement selected from the group A279T (replacement of alanine at position 279 of the coded aspartate kinase protein according to SEQ ID NO: 8 by threonine), S381F (replacement of serine at position 891 of the coded aspartate kinase protein according to SEQ ID NO: 8 by phenylalanine), where the serine at position 317 is replaced, if appropriate, by alanine (S317A).

Very particular preference is given to the lysC^{FBR} allele lysC T311I (replacement of threonine at position 311 of the coded aspartate kinase protein according to SEQ ID NO: 8 by isoleucine), where, if appropriate, the serine at position 317 is replaced by alanine (S317A).

In the case of the Enterobacteriaceae, the genus Escherichia is preferred. In the case of the genus Escherichia, strains of the species Escherichia coli are preferred.

Known representatives of L-lysine-excreting strains of the species Escherichia coli are, for example:
Escherichia coli pDA1/TOC21R (=CNCM I-167) described in FR-A-2511032,
Escherichia coli NRRL B-12199 described in U.S. Pat. No. 4,346,170 and
Escherichia coli NRRL B-12185 described in U.S. Pat. No. 4,346,170.

In the case of the genus Bacillus, L-lysine-excreting strains of the species Bacillus subtilis and Bacillus methanolicus are preferred as are described, for example, in U.S. Pat. No. 6,110,713, U.S. Pat. No. 6,261,825, U.S. Pat. No. 7,160,704 and U.S. Pat. No. 7,163,810.

Strains having the designation “ATCC” can be obtained from the American Type Culture Collection (Manassas, Va., USA). Strains having the designation “DSM” can be obtained from the Deutsche Sammlung von Mikroorganismen und Zellkulturen [German collection of microorganisms and cell cultures] (DSMZ, Brunswick, Germany). Strains having the designation “FERM” can be obtained from the National Institute of Advanced Industrial Science and Technology (AIST Tsukuba Central 6, 1-1-1 Higashi, Tsukuba Ibaraki, Japan). Strains having the designation “CNCM” can be obtained from the Collection Nationale de Cultures de Microorganismes [National collection of microorganism cultures] at the Institut Pasteur (Paris, France).

A gene or allele is chemically a polynucleotide. Another name for this is nucleic acid, in particular deoxyribonucleic acid.

The terms polypeptide and protein are mutually exchangeable.

A polypeptide having LL-diaminopimelate aminotransferase activity is taken to mean an enzyme which catalyzes establishment of equilibrium between L-2,3,4,5-tetradihydrodipicolinic acid (THDPA) and LL-2,5-diaminopimelic acid (LL-DAP) using L-glutamic acid and a-ketoglutaric acid as cosubstrates.

The polynucleotides used for the measures of the invention code for polypeptides having an amino acid sequence which is at least 70%, or at least 80%, preferably at least 90% or at least 95%, particularly preferably at least 96%, or at least 97%, or at least 98% and very particularly preferably at least 99% identical to SEQ ID NO: 2, SEQ ID NO: 4, SEQ ID NO: 6 or SEQ ID NO: 10 and have LL-diaminopimelate aminotransferase activity. Preferably, the coded polypeptides have a length corresponding to 412 amino acids as in SEQ ID NO: 4 or a length corresponding to 461 amino acids as in SEQ ID NO: 2 or a length corresponding to 464 amino acids as shown in SEQ ID NO: 6 or a length of 459 amino acids as shown in SEQ ID NO: 10.

For the measures of the invention, use can likewise be made of polynucleotides which code for polypeptides having LL-diaminopimelate aminotransferase activity which comprise or possess the amino acid sequence of SEQ ID NO: 2, SEQ ID NO: 4, SEQ ID NO: 6 or SEQ ID NO: 10 including one or more of the features selected from the group
a) one (1) conservative or a plurality of conservative amino acid replacement(s),
b) an extension at the N- or C-terminus by one (1) or a plurality of amino acid(s), and
c) one (1) or a plurality of insertions or deletions of one (1) or a plurality of amino acid(s).

Preferably, the number of conservative amino acid replacements is at most five (5), at most four (4), at most three (3), or at most two (2).

In the case of the aromatic amino acids, conservative replacements are taken to mean when phenylalanine, tryptophan and tyrosine are exchanged for one another. In the case of the hydrophobic amino acids, conservative replacements are taken to mean when leucine, isoleucine and valine are exchanged for one another. In the case of the polar amino acids, conservative replacements are taken to mean when glutamine and asparagine are exchanged for one another. In the case of the basic amino acids, conservative replacements are taken to mean when arginine, lysine and histidine are exchanged for one another. In the case of the acidic amino acids, conservative replacements are taken to mean when aspartic acid and glutamic acid are exchanged for one another. In the case of the hydroxyl-containing amino acids, conservative replacements are taken to mean when serine and threonine are exchanged for one another.

Preferably, the extension at the N- or C-terminus of the polypeptide is no more than 50, 40, 30, 20, 10, 5, 3 or 2 amino acids.

Preferably, the number of insertions or deletions within the polypeptide is a maximum of 5, a maximum of 4, a maximum of 3, or a maximum of 2, with respectively a maximum of 10, a maximum of 5, a maximum of 4, a maximum of 3, a maximum of 2 inserted or deleted amino acids or a maximum of 1 inserted or deleted amino acid per insertion or deletion.

For the measures of the invention, finally, use can also be made of polynucleotides which code for polypeptides having LL-diaminopimelate aminotransferase activity which comprise or possess the amino acid sequence of SEQ ID NO: 2, SEQ ID NO: 4, SEQ ID NO: 6 or SEQ ID NO: 10.

The polynucleotide is subsequently expressed in the chosen host.

In the prior art, for expression of the polynucleotide in coryneform bacteria, numerous promoters are described which enable a desired concentration or activity of the polypeptide or of the enzyme to be set. For example, the lysC promoter of the mutant DM58-1 which is described in Kalinowski et al. (Molecular Microbiology 5(5), 1197-1204 (1991)), or the gap promoter which is described in the patent application having the application number EP 06007373.1, can be used. In addition, use can be made of the promoters described in the patent application having the application number EP 06117294.6 which are described in the journal Research Disclosure (contribution 512057 in the edition of December 2006, pages 1616 to 1618), the promoters described by Patek et al. (Journal of Biotechnology 104(1-3), 311-323 (2003)), or the variants of the dapA promoter, for example the promoter A25, described by Vasicova et al. (Journal of Bacteriology 181, 6188-6191 (1999)).

In the same manner, use can also be made of the promoters known from Escherichia coli genetics such as, for example, tac promoter, trp promoter, trc promoter and lpp promoter, or the P_{L} and P_{R} promoter of phage λ.

The polynucleotide provided in such a manner with a promoter can be incorporated in the form of one (1) or a plurality of copies into the desired coryneform bacterium.

For this, use can be made of, for example, plasmids which are replicated from coryneform bacterium. Suitable plasmid vectors are, for example, pZ1 (Menkel et al., Applied and Environmental Microbiology (1989) 64: 549-554) or the PSELF vectors described by Tauch et al. (Journal of Biotechnology 99, 79-91 (2002)). A review article on the topic of plasmids in Corynebacterium glutamicum may be found under Tauch et al. (Journal of Biotechnology 104, 27-40 (2003)).

In addition, a copy of the polynucleotide can be introduced into the chromosome of a coryneform bacterium.

In an embodiment, a plasmid which is non-replicative in coryneform bacteria is used. Firstly the gene of interest coding for a polypeptide having LL-diaminopimelate aminotransferase activity, and secondly a DNA fragment from a coryneform bacterium are incorporated into this plasmid. Said DNA fragment represents the destination within the chromosome of the coryneform bacterium in which the corresponding plasmid is integrated by homologous recombination. Suitable destinations for Corynebacterium glutamicum are described, inter alia, in Table 3 of WO 03/040373 and in Tables 12 and 13 of WO 04/069996. After conjugation or transformation and homologous recombination by means of a cross-over event, the resultant strain contains the corresponding plasmid and consequently at least two copies of the gene in question.

In a further embodiment, use is likewise made of a plasmid which is non-replicative in coryneform bacteria. Here, a DNA fragment which represents the destination within the chromosome of the coryneform bacterium into which the corresponding gene is integrated by homologous recombination is attached at the 5′ terminal and the 3′ terminal of the desired gene coding for a polypeptide having LL-diaminopimelate aminotransferase activity. Suitable destinations for Corynebacterium glutamicum are, inter alia, described in Table 3 of WO 03/040373, in tables 12 and 13 of WO 04/069996, in Aham et al. (Applied and Environmental Microbiology 67(12), 5425-5430 (2001), or Correia et al. (FEMS Microbiology Letters 142, 259-264 (1996)). After conjugation or transformation and homologous recombination by means of at least two recombination events, the resultant strain contains at least two copies of the gene in question.

In a further embodiment which is described in WO 03/014330 and US-2004-0043458-A1, a tandem-duplication of the gene can be achieved following integration into the chromosome.

Finally, it is possible to set the copy number of the gene using transposons or IS elements (see: U.S. Pat. No. 5,804,414 or U.S. Pat. No. 5,591,577).

The polynucleotide is expressed in Enterobacteriaceae as in the prior art as described in, for example, Makrides et al. (Microbiological Reviews 60 (3), 512-538 (1996)).

The content of LL-diaminopimelate aminotransferase activity achieved by the expression measures can be determined using an enzyme test, as described in Hudson et al. (Plant Physiology 140, 292-301 (2006)).

The concentration of the protein can be determined by 1- and 2-dimensional protein gel separation and subsequent optical identification of the protein concentration using appropriate evaluation software in the gel. A customary method of preparing protein gels in the case of coryneform bacteria and for identifying proteins is the procedure described by Hermann et al. (Electrophoresis, 22: 1712-23 (2001)). The protein concentration can likewise be determined by Western-blot hybridization using an antibody specific for the protein to be detected (Sambrook et al., Molecular cloning: a laboratory manual. 2nd Ed. Cold Spring Harbor Laboratory Press, Cold Spring Harbor, N.Y., 1989) and subsequent optical evaluation with corresponding software for determination of concentration (Lohaus and Meyer (1998) Biospektrum 5: 32-39; Lottspeich, Angewandte Chemie 111: 2630-2647 (1999)).

The invention correspondingly relates to a bacterium excreting L-lysine which has been transformed by a polynucleotide, coding for a polypeptide having LL-diaminopimelate aminotransferase activity, and, compared with the non-transformed bacterium, exhibits an increased excretion of L-lysine.

In a further embodiment, the coryneform bacteria used for the measures of the invention possess one or more of the features, selected from the group
a) overexpressed polynucleotide (dapA gene) which codes for a dihydrodipicolinate synthase (DapA, EC No. 4.2.1.52),
b) overexpressed polynucleotide (asd gene) which codes for an aspartate semialdehyde dehydrogenase (Asd, EC No. 1.2.1.11),
c) overexpressed polynucleotide (lysA gene) which codes for a diaminopimelate decarboxylase (LysA, EC No. 4.1.1.20),
d) overexpressed polynucleotide (aat gene) which codes for an aspartate aminotransferase (Aat, EC No. 2.6.1.1),
e) overexpressed polynucleotide (lysE gene) which codes for a polypeptide having L-lysine export activity (LysE, lysine efflux permease),
f) overexpressed polynucleotide (pyc gene) which codes for a pyruvate carboxylase (Pyc, EC No. 6.4.1.1),
g) overexpressed polynucleotide (lysC^{FBR} allele) which codes for a feedback resistant aspartate kinase (LysC^{FBR})
h) overexpressed polynucleotide (dapF gene) which codes for a diaminopimelate epimerase (DapF, EC No. 5.1.1.7),
i) overexpressed polynucleotide (dapB gene), which codes for a dihydrodipicolinate reductase (DapB, 1.3.1.26),
j) overexpressed polynucleotide (zwf gene), which codes for the Zwf subunit of glucose 6-phosphate dehydrogenase (Zwf, EC. No. 1.1.1.49),
k) overexpressed polynucleotide (opcA gene), which codes for the OpcA subunit of glucose 6-phosphate dehydrogenase (OpcA, EC. No. 1.1.1.49),
l) overexpressed polynucleotide (gnd gene), which codes for the phosphogluconic acid dehydrogenase (Gnd, EC, No. 1.1.1.44),
m) switched-off or attenuated activity of malate quinone oxidoreductase (Mqo, EC No. 1.1.99.16) and
n) switched-off or attenuated activity of the E1p subunit of pyruvate dehydrogenase complex (AceE, EC No. 1.2.4.1),
o) switched-off or attenuated activity of citrate synthase (GltA, EC No. 4.1.3.7),
p) switched-off or attenuated activity of malate dehydrogenase (Mdh, EC No. 1.1.1.37), and
q) switched-off or attenuated activity of the UDP N-acetylmuramoylalanyl-D-glutamate-2,6-diamino-pimelate ligase, 6-diaminopimelate ligase (MurE, EC No. 6.3.2.13).

Preference is given to overexpression of one or more of the polynucleotides selected from the group consisting of the polynucleotide coding for a feedback-resistant aspartate kinase (LysC^{FBR}), the polynucleotide coding for a diaminopimelate decarboxylase (LysA), the polynucleotide coding for a diaminopimelate epimerase (DapF) and the polynucleotide coding for a dihydrodipicolinate synthase (DapA).

Particular preference is given to overexpression of one or more of the polynucleotides selected from the group consisting of the polynucleotide coding for a feedback-resistant aspartate kinase (LysC^{FBR}), the polynucleotide coding for a diaminopimelate decarboxylase (LysA) and the polynucleotide coding for a diaminopimelate epimerase (DapF).

For overexpression of the genes, or polynucleotides, listed, use can be made of the genes known in the prior art, for example what are termed the wild type genes, of Corynebacterium glutamicum, Corynebacterium efficiens, Escherichia coli (Blattner et al., Science 277(5), 1453-1462 (1997)), Bacillus subtilis (Kunst et al, Nature 390 (6657), 249-256 (1997)), Bacillus licheniformis (Veith et al, Journal of Molecular Microbiology and Biotechnology 7(4), 204-211 (2004)), Mycobacterium tuberculosis (Fleischmann et al, Journal of Bacteriology 1841, 5479-5490 (2004)), Mycobacterium bovis (Garnier et al, Proceedings of the National Academy of Sciences U.S.A. 100 (13), 7877-7882 (2003)), Streptomyces coeliclor (Redenbach et al, Molecular Microbiology 21 (1), 77-96 (1996)), Lactobacillus acidophilus (Altermann et al, Proceedings of the National Academy of Sciences U.S.A. 102 (11), 3906-3912 (2005)), Lactobacillus johnsonii (Pridmore et al, Proceedings of National Academy of Sciences U.S.A. 101 (8), 2512-2517 (2004)), Bifidobacterium longum (Schell et al, Proceedings of National Academy of Sciences U.S.A. 99 (22), 14422-14427 (2002)), and Saccharomyces cerevisiae. The genomes of the wild type forms of these bacteria are available in sequenced and annotated form. Preferably, use is made of the genes, or polynucleotides, of the genus Corynebacterium, particularly preferably of the species Corynebacterium glutamicum.

The dapA gene of Corynebacterium glutamicum strain ATCC13032 is described, for example, in EP 0 197 335. For overexpression of the dapA gene of Corynebacterium glutamicum, in addition, use can be made, inter alia, of the mutations MC20 and MA16 of the dapA promoter, as described in U.S. Pat. No. 6,861,246.

The asd gene of Corynebacterium glutamicum strain ATCC21529 is described, for example, in U.S. Pat. No. 6,927,046.

The lysA gene of Corynebacterium glutamicum ATCC13869 (Brevibacterium lactofermentum) is described, for example, in U.S. Pat. No. 6,090,597.

The aat gene of Corynebacterium glutamicum ATCC13032 is described, for example, in Kalinowski et al. (Journal of Biotechnology 104 (1-3), 5-25 (2003); see also accession number NC_{—}006958). There it is termed aspB gene. In U.S. Pat. No. 6,004,773, a gene coding for an aspartate aminotransferase is termed aspC. Marienhagen et al. (Journal of Bacteriology 187 (22), 7693-7646 (2005)) designate the aat gene and aspT gene.

The lysE gene of Corynebacterium glutamicum R127 is described, for example, in U.S. Pat. No. 6,858,406. Strain R127 is a restriction-defective mutant of ATCC13032 (Liebl et al, FEMS Microbiology Letters 65, 299-304 (1989)). In the same manner, the LysE gene of strain ATCC13032 used in U.S. Pat. No. 6,861,246 can be used.

The pyc gene of Corynebacterium glutamicum of strain ATCC13032 is described, for example, in WO 99/18228 and WO 00/39305. In addition, use can be made of alleles of the pyc gene as described, for example, in U.S. Pat. No. 6,965,021. The pyruvate carboxylases described in this patent possess one or more of the amino acid replacements selected from the group: Pyc E153D (replacement of L-glutamic acid at position 153 by L-aspartic acid), Pyc A182S (replacement of L-alanine at position 182 by L-serine), Pyc A206S (replacement of L-alanine at position 206 by L-serine), Pyc H227R (replacement of L-histidine at position 227 by L-arginine), Pyc A455G (replacement of L-alanine at position 455 by glycine), and Pyc D1120E (replacement of L-aspartic acid at position 1120 by L-glutamic acid). In the same manner, the pyc allele described in EP 1 108 790 can be used which codes for a pyruvate carboxylase which contains the amino acid replacement Pyc P458S (replacement of L-proline at position 458 by L-serine).

The dapF gene of Corynebacterium is described, for example, in EP 1085094.

For the measures of overexpression of said genes, the same measures can be used as for the expression of the LL-diaminopimelate aminotransferase gene.

By means of the measures of overexpression, the activity or concentration of the corresponding polypeptide is generally increased by at least 10%, 25%, 50%, 75%, 100%, 150%, 200%, 300%, 400% or 500%, maximally up to 1000% or 2000%, based on the activity or concentration of the polypeptide in the strain (starting strain, parent strain) before the measure leading to overexpression.

Switched-off or attenuated activity is taken to mean the reduction or switching off of the intracellular activity or concentration of one or more enzymes or proteins in a microorganism which are coded for by the corresponding polynucleotide or DNA.

To generate a strain in which the intracellular activity of a desired polypeptide is switched off, a deletion or insertion of at least one (1) nucleobase, preferably one (1) or two (2) nucleobases, is built into the coding region of the corresponding gene. It is likewise possible to delete at least one (1) or else a plurality of codon(s) within the coding region. These measures lead to a shifting of the reading frame (frame shift mutations) and thus typically to the synthesis of a non-functional polypeptide. Introducing a nonsense mutation by transversion or transition of at least one (1) nucleobase within the coding region acts in the same manner. Owing to the resultant stop codon, translation terminates prematurely. Said measures are preferably carried out in the 5′-terminal part of the coding region, which codes for the N-terminus of the polypeptide. Designating the total length of a polypeptide (measured as the number of chemically bound L-amino acids) as 100%, in the context of the present invention, the N-terminus of the polypeptide encompasses the part of the amino acid sequence which, calculated from the start amino acid L-formylmethionine, contains 80% of the following L-amino acids.

Genetic measures for switching off the malate-quinone oxidoreductase (Mqo) are described, for example, in U.S. Pat. No. 7,094,106.

Genetic measures for switching off the malate dehydrogenase (Mdh) are described, for example, in WO 02/02778.

Genetic measures for switching off the E1p subunit (AceE) of the pyruvate dehydrogenase complex are described, for example, in EP1767616 and in Schreiner et al. (Journal of Bacteriology 187(17), 6005-6018 (2005)).

It is likewise possible to lower the catalytic property of the polypeptide in question by suitable amino acid replacements.

In the case of malate-quinone oxidoreductase (Mqo) this can be achieved, as described in PCT/EP2005/057216, by using alleles of the mqo gene which code for an Mqo variant which contains one or more amino acid replacements selected from the group replacement of the L-serine at position 111 by another proteinogenic amino acid, preferably L-phenylalanine or L-alanine, and replacement of the L-alanine at position 201 by another proteinogenic amino acid, preferably L-serine.

In the case of the citrate synthase (GltA) this can be achieved as described in PCT/EP2007/056153 by using alleles of the gltA gene coding for a GltA variant in which the L-aspartic acid at position 5 of the amino acid sequence is replaced by another proteinogenic amino acid, preferably L-valine, L-leucine and L-isoleucine, particularly preferably L-valine. Finally, by deleting the promoter region of the gltA gene of Corynebacterium glutamicum which comprises positions 664 to 822 of SEQ ID NO: 11, a reduction of the expression of citrate synthase of about 90% is achieved.

It is also possible to achieve attenuation of the expression of a desired gene by replacing the start codon ATG of the coding region by GTG or TTG.

Finally, attenuation of expression of a desired gene can be achieved by using so-called weak promoters. To this end, it is possible to use, for example, the variants of the dapA promoter C13, O1, C2, J2, B31, C5 and B6 described by Patek in “Handbook of Corynebacterium glutamicum” (Lothar Eggeling and Michael Bott (editors), CRC Press, Taylor and Francis Group, Boca Raton, Fla., USA, 2005). Furthermore, it is possible to use the weak promoters employed in the publication Research Disclosure in the article having the number RD 512057.

The switching-off or attenuation measures reduced the activity or concentration of the corresponding protein generally to 0 to 75%, 0 to 50%, 0 to 25%, 0 to 10% or 0 to 5% of the activity or concentration of the wild type protein, or of the activity or concentration of the protein in the starting strain or parent strain.

The bacteria modified in such a manner are subsequently cultured or fermented in a medium for a desired time.

The performance of the bacteria modified in such a manner or the fermentation process using the modified bacteria with respect to one or more of the parameters selected from the group product concentration (product per unit volume), product yield (product formed per amount of carbon source consumed) and product formation (product formed per unit volume and time) or else other process parameters and combinations thereof is improved by at least 0.5%, at least 1%, at least 1.5% or at least 2%, based on the starting strain or parent strain or on the fermentation process using the same.

The bacteria can be cultured continuously, as described, for example, in PCT/EP2004/008882, or discontinuously in the batch process (batch culture) or in the fed-batch process or repeated fed-batch process for the purpose of the production of L-amino acids. A summary of a general type of known culture methods is available in the textbook by Chmiel (Bioprozesstechnik 1. Einführung in die Bioverfahrenstechnik [Bioprocess technology 1. Introduction to bioengineering technology] (Gustav Fischer Verlag, Stuttgart, 1991)) or in the textbook by Storhas (Bioreaktoren und periphere Einrichtungen [Bioreactors and peripheral equipment] (Vieweg Verlag, Brunswick/Wiesbaden, 1994)).

The culture medium or fermentation medium to be used must satisfy in a suitable manner the demands of the respective strains. Descriptions of culture media of various microorganisms are contained in the handbook “Manual of Methods for General Bacteriology” of the American Society for Bacteriology (Washington D.C., USA, 1981). The expressions culture medium, fermentation medium and nutrient medium or medium are mutually interchangeable.

As carbon source, use can be made of sugar and carbohydrates such as, for example, glucose, sucrose, lactose, fructose, maltose, molasses, sucrose-containing solutions from sugar beet or sugar cane production, starch, starch hydrolyzate and cellulose, oils and fats, such as, for example, soybean oil, sunflower oil, peanut oil and coconut fat, fatty acids such as, for example, palmitic acid, stearic acid and linoleic acid, alcohols such as, for example, glycerol, methanol and ethanol, and organic acids such as, for example, acetic acid. These substances can be used individually or as a mixture.

As nitrogen source, use can be made of organic nitrogenous compounds such as peptones, yeast extract, meat extract, malt extract, corn steep liquor, soybean meal and urea or inorganic compounds such as ammonium sulfate, ammonium chloride, ammonium phosphate, ammonium carbonate and ammonium nitrate. The nitrogen sources can be used individually or as a mixture.

As phosphorus source, use can be made of phosphoric acid, potassium dihydrogenphosphate or dipotassium hydrogenphosphate or the corresponding sodium-containing salts.

The culture medium must in addition contain salts, for example in the form of chlorides or sulfates of metals such as, for example sodium, potassium, magnesium, calcium and iron, such as, for example, magnesium sulfate or iron sulfate, which are essential for growth. Finally, essential growth substances such as amino acids, for example homoserine, and vitamins, for example thiamine, biotin or pantothenic acid, can be used in addition to the abovementioned substances. In addition, suitable precursors of the respective amino acid can be added to the culture medium.

Said starting materials can be added to the culture in the form of a single batch, or suitably fed during the culture.

For pH control of the culture, use is made of basic compounds such as sodium hydroxide, potassium hydroxide, ammonia or ammonia water or acidic compounds such as phosphoric acid or sulfuric acid in a suitable manner. The pH is generally set to a value of 6.0 to 9.0, preferably 6.5 to 8. To control foam development, use can be made of antifoams, such as for example fatty acid polyglycol esters. To maintain the stability of plasmids, suitable selectively acting substances can be added to the medium, such as, for example antibiotics. To maintain aerobic conditions, oxygen or oxygen-containing gas mixtures such as, for example air, are introduced into the culture. The use of liquids which are enriched with hydrogen peroxide is likewise possible. If appropriate, the fermentation is run at superatmospheric pressure, for example at a pressure of 0.03 to 0.2 MPa. The temperature of the culture is usually 20° C. to 45° C., and preferably 25° C. to 40° C. In batch processes the culture is continued until a maximum of L-lysine has formed. This target is usually achieved in the course of 10 hours to 160 hours. In continuous processes, longer culture times are possible.

Suitable fermentation media are described, inter alia, in U.S. Pat. No. 6,221,636, in U.S. Pat. No. 5,840,551, in U.S. Pat. No. 5,770,409, in U.S. Pat. No. 5,605,818, in U.S. Pat. No. 5,275,940, in U.S. Pat. No. 4,275,157 and in U.S. Pat. No. 4,224,409 and also in U.S. Pat. No. 5,989,875.

Methods for determining L-lysine are known from the prior art.

Analysis of L-lysine for determining the concentration at one or more point(s) of time during the course of the fermentation can be carried out by separating the L-amino acids by ion exchange chromatography, preferably by cation exchange chromatography with subsequent post-column derivatization using ninhydrin, as described by Spackman et al. (Analytical Chemistry 30: 1190-1206 (1958)). Instead of ninhydrin, it is also possible to use ortho-phthadialdehyde for post-column derivatization. A review article of ion exchange chromatography can be found in Pickering (LC•GC (Magazine of Chromatographic Science) 7(6), 484-487 (1989)).

It is also possible to carry out a pre-column derivatization using, for example, ortho-phthadialdehyde or phenyl isothiocyanate, and separating the amino acid derivatives formed by reverse-phase chromatography (RP), preferably in the form of high-performance liquid chromatography (HPLC). Such a method is described, for example, in Lindroth et al. (Analytical Chemistry 51: 1167-1174 (1979)).

Detection is carried out photometrically (absorption, fluorescence).

A comprehensive text on amino acid analysis can be found, inter alia, in the textbook “Bioanalytik” by Lottspeich and Zorbas (Spektrum Akademischer Verlag, Heidelberg, Germany 1998). The analysis can proceed, for example as described in Spackman et al. (Analytical Chemistry, 30, (1958), 1190) by anion-exchange chromatography with subsequent ninhydrin derivatization, or it can proceed via reversed-phase HPLC, as described in Lindroth et al. (Analytical Chemistry (1979) 51: 1167-1174).

The fermentation is generally followed by collection of the L-lysine accumulated in the fermentation broth, that is in the medium and/or in the cells of the bacteria, in order to arrive at a solid or liquid product.

A fermentation broth is considered to mean a fermentation medium in which a microorganism has been cultured for a certain time at a certain temperature. The fermentation medium or the media used during the fermentation contains/contain all substances or components which ensure multiplication of the microorganism and formation of the desired amino acid.

On completion of the fermentation, the resultant fermentation broth accordingly contains a) the biomass (cell mass) of the microorganism resulting from multiplication of the cells of the microorganism, b) the L-lysine formed in the course of the fermentation, c) the organic byproducts formed in the course of the fermentation and d) the components unused in fermentation of the fermentation medium/fermentation media or starting materials such as, for example vitamins such as biotin, amino acids such as homoserine, or salts such as magnesium sulfate used.

The organic byproducts encompass substances which are produced by the microorganisms used in the fermentation in addition to the desired L-lysine and are if appropriate excreted. These include proteinogenic L-amino acids which, compared with the desired L-lysine, make up less than 30%, 20% or 10%. These further encompass organic acids which bear 1 to 3 carboxyl groups, for example acetic acid, lactic acid, citric acid, malic acid or fumaric acid. Finally, sugars are also encompassed, such as, for example, trehalose.

Proteinogenic amino acids are taken to mean generally the amino acids which occur in natural proteins, that is in proteins of microorganisms, plants, animals and humans. In connection with the present invention, proteinogenic amino acids is taken to mean the group of L-amino acids consisting of L-aspartic acid, L-asparagine, L-threonine, L-serine, L-glutamic acid, L-glutamine, glycine, L-alanine, L-cysteine, L-valine, L-methionine, L-isoleucine, L-leucine, L-tyrosine, L-phenylalanine, L-histidine, L-lysine, L-tryptophan, L-proline and L-arginine and if appropriate L-selenocysteine.

Typical fermentation broths suitable for industrial purposes typically have an amino acid content of 30 g/kg to 200 g/kg, or 40 g/kg to 175 g/kg, or 50 g/kg to 150 g/kg. The biomass content (as dried biomass) is generally 20 to 50 g/kg.

L-Lysine is known in the prior art essentially in four different product forms.

One group of L-lysine-containing products comprises concentrated, aqueous, alkaline solutions of purified L-lysine (EP-B-0534865). A further group as described, for example, in U.S. Pat. No. 6,340,486 and U.S. Pat. No. 6,465,025, comprises aqueous, acidic, biomass-containing concentrates of L-lysine-containing fermentation broths. The best known group of solid products comprises pulverulent or crystalline forms of purified or pure L-lysine which is typically in the form of a salt such as, for example, L-lysine monohydrochloride. A further group of solid product forms is described, for example, in EP-B-0533039. The product form described there, in addition to L-lysine, contains the majority of the starting materials used and not consumed during the production by fermentation and if appropriate the biomass of the microorganism used at a fraction of >0%-100%.

In accordance with the various product forms, the most varied processes are known in which the L-lysine is collected, isolated or purified from the fermentation broth in order to produce the L-lysine-containing product or the purified L-lysine.

To produce solid, pure L-lysine, essentially use is made of methods of ion-exchange chromatography if appropriate using activated carbon and methods of crystallization. This produces the corresponding base or a corresponding salt such as, for example, the monohydrochloride (Lys-HCl) or lysine sulfate (Lys₂-H₂SO₄), for example in a purity of =95% by weight or =98% by weight.

EP-B-0534865 describes a process for producing aqueous, basic L-lysine-containing solutions from fermentation broths. In the process described there, the biomass is separated off from the fermentation broth and discarded. By means of a base such as, for example, sodium hydroxide, potassium hydroxide or ammonium hydroxide, a pH between 9 and 11 is set. The mineral components (inorganic salts), after concentration and cooling, are separated off from the broth by crystallization and either used as fertilizer or discarded.

In processes for producing L-lysine using the bacteria produced, use is also made of such processes in which products are obtained which contain components of the fermentation broth, for example biomass. These products are used in particular as animal feed additives.

Depending on requirements, the biomass can be completely or partially removed from the fermentation broth by separation methods such as, for example, centrifugation, filtration, decanting, or a combination thereof, or left completely in the fermentation broth. If appropriate, the biomass or the biomass-containing fermentation broth is inactivated in the course of a suitable process step, for example by thermal treatment (heating) or by acid addition.

The chemical components of the biomass are, inter alia, the cell envelope, for example the peptidoglycan and the arabinogalactan.

In one procedure, the biomass is completely or virtually completely removed, so that none (0%), or at most 30%, at most 20%, at most 10%, at most 5%, at most 1% or at most 0.1% biomass remains in the product produced. In a further procedure, the biomass is not removed or removed only in minor fractions, so that all (100%) or more than 70%, 80%, 90%, 95%, 99% or 99.9% biomass remains in the product produced. In a process according to the invention, accordingly the biomass is removed in fractions=0% to =100%.

Finally, the fermentation broth obtained after the fermentation, before or after complete or partial removal of the biomass, can be set to an acidic pH using an inorganic acid such as, for example, hydrochloric acid, sulfuric acid, or phosphoric acid, or organic acid such as, for example, propionic acid (GB 1,439,728 or EP 1 331 220). It is equally possible to acidify the fermentation broth containing the complete biomass (U.S. Pat. No. 6,340,486 or U.S. Pat. No. 6,465,025). Finally, the broth can also be stabilized by addition of sodium bisulfite (NaHSO₃, GB 1,439,728) or another salt, for example the ammonium, alkali metal or alkaline earth metal salt of sulfurous acid.

When the biomass is separated off, organic or inorganic solids possibly present in the fermentation broth are partially or completely removed. The dissolved organic byproducts in the fermentation broth and the dissolved unused components of the fermentation medium (starting materials) remain at least in part (>0%), preferably at at least 25%, particularly preferably at least 50%, and very particularly preferably at least 75% in the product. If appropriate these also remain completely (100%) or virtually completely, that is >95% or >98%, in the product. In this context the expression “fermentation broth base” means that a product contains at least a part of the components of the fermentation broth.

Subsequently the broth is dewatered using known methods such as, for example, using a rotary evaporator, thin-film evaporator, falling-film evaporator, by reverse osmosis or by nanofiltration or it is thickened or concentrated. This concentrated fermentation broth can subsequently be worked up by methods of freeze drying, spray drying, spray granulation or by other methods such as, for example, in the circulating fluidized bed described according to PCT/EP2004/006655, to give free-flowing products, in particular to give a finely divided powder or, preferably coarse-grained granules. If appropriate a desired product is isolated from the resultant granules by sieving or dust removal.

It is likewise possible to dry the fermentation broth directly, that is without previous concentration, by spray drying or spray granulation.

“Free-flowing” is taken to mean powders which flow unimpeded out of a series of glass outlet vessels having differently sized outlet openings, at least from the vessel having the opening of 5 mm (millimeters) (Klein: Seifen, Óle, Fette, Wachse 94, 12 (1968)).

“Finely divided” means a powder having a predominant fraction (>50%) of a particle size of 20 to 200 μm in diameter.

“Coarse-grained” means a product having a predominant fraction (>50%) of a particle size of 200 to 2000 μm in diameter.

Particle size can be determined by methods of laser diffraction spectrometry. The corresponding methods are described in the textbook on “Teilchengröβenmessung in der Laborpraxis” [Particle size measurement in laboratory practice] by R. H. Müller and R. Schuhmann, Wissenschaftliche Verlagsgesellschaft Stuttgart (1996) or in the textbook “Introduction to Particle Technology” by M. Rhodes, Verlag Wiley & Sons (1998).

The free-flowing, finely divided powder can be converted in turn by suitable compacting or granulation processes into a coarse-grained, readily free-flowing, storable and substantially dust-free product.

The expression “dust-free” means that the product contains only small fractions (<5%) of particle sizes less than 100 μm in diameter.

“Storable” in the context of this invention means a product which can be stored at least one (1) year or longer, preferably at least 1.5 years or longer, particularly preferably two (2) years or longer in dry and cool surroundings, without significant loss (<5%) of the respective amino acid occurring.

The invention further relates, correspondingly, to a process for producing an L-lysine-containing product, preferably animal feed additive, from fermentation broths, characterized by the steps
a) culturing and fermentation in a fermentation medium of a L-lysine-excreting bacterium in which a polynucleotide coding for a polypeptide having LL-diaminopimelate aminotransferase activity is expressed,
b) after the fermentation has ended, removing the biomass formed during the fermentation in an amount of 0 to 100% by weight and
c) drying the fermentation broth obtained according to a) and/or b) and conversion into the desired powder or granule form,
   if appropriate before step b) or c) an acid selected from the group sulfuric acid, phosphoric acid or hydrochloric acid being added. Preferably, subsequently to step a) or b), water is removed from the L-lysine-containing fermentation broth (concentration).

The invention further relates to a process for producing a lysine-sulfate-containing product which is described in its basics in DE 102006016158, and in which the fermentation broth obtained using the bacteria produced according to the invention, from which the biomass, if appropriate had been completely or partially separated off, is further processed by carrying out a process which comprises at least the following steps:
a) lowering the pH by addition of sulfuric acid to 4.0 to 5.2, in particular 4.9 to 5.1, and setting a sulfate/L-lysine molar ratio of 0.85 to 1.2, preferably 0.9 to 1.0, particularly preferably >0.9 to <0.95, in the broth, if appropriate by addition of a further or a plurality of sulfate-containing compound(s) and
b) concentrating the resultant mixture by dewatering and if appropriate granulating it,
   with if appropriate, before step a), one or both of the following measures being carried out:
c) measuring the molar ratio of sulfate/L-lysine for determining the required amount of sulfate-containing compound(s)
d) adding a sulfate-containing compound selected from the group ammonium sulfate, ammonium hydrogen sulfate and sulfuric acid in corresponding ratios.

If appropriate, in addition before step b) a salt of sulfurous acid is added, preferably alkali metal hydrogen sulfate particularly preferably sodium hydrogen sulfate in a concentration of 0.01 to 0.5% by weight, preferably 0.1 to 0.3% by weight, particularly preferably 0.1 to 0.2% by weight, based on the fermentation broth.

Preferred sulfate-containing compounds in the context of the abovementioned method steps are, in particular, ammonium sulfate and/or ammonium hydrogen sulfate or corresponding mixtures of ammonia and sulfuric acid and sulfuric acid itself.

The molar sulfate/L-lysine ratio V is calculated from the formula: V=2×[SO₄²⁻]/[L-lysine]. This formula takes into account the fact that the SO₄²⁻ anion is divalent. A ratio V=1 means that a stoichiometrically composed Lys₂ (SO₄) is present, whilst at a ratio of V=0.9, a 10% sulfate deficit is found, and at a ratio of V=1.1, a 10% sulfate excess is found.

It is advantageous in the granulation or compacting to make use of customary organic or inorganic auxiliaries or supports such as starch, gelatin, cellulose derivatives or similar materials, as are customarily used in food or feed processing as binder, gelling agent or thickener or other substances such as, for example, silicas, silicates (EP 0743016A) or stearates.

In addition it is advantageous to provide the surface of the resultant granules with oils as described in WO 04/054381. Oils which can be used are mineral oils, vegetable oils or mixtures of vegetable oils. Examples of such oils are soybean oil, olive oil, soybean oil/lecithin mixtures. In the same manner, silicone oils, polyethylene glycols or hydroxyethylcellulose are also suitable. Treatment of the surfaces with said oils achieves an increased abrasion resistance of the product and a decrease of the dust fraction. The oil content in the product is 0.02 to 2.0% by weight, preferably 0.02 to 1.0% by weight, and very particularly preferably 0.2 to 1.0% by weight, based on the total amount of the feed additive.

Preference is given to products having a fraction of 97% by weight of a particle size from 100 to 1800 μm, or a fraction of 95% by weight of a particle size from 300 to 1800 μm in diameter. The fraction of dust, that is particles having a particle size <100 μm is preferably >0 to 1% by weight, particularly preferably maximally 0.5% by weight.

Alternatively, the product, can also be taken up onto an organic or inorganic carrier and customary in food processing such as, for example, silicas, silicates, coarse meals, brans, flours, starches, sugars or others and/or is mixed and stabilized with customary thickeners or binders. Examples and methods for this are described in the literature (Die Mühle+Mischfuttertechnik 132 (1995) 49, page 817).

Finally, the product may also, by means of coating processes using film-forming agents such as, for example, metal carbonates, silicas, silicates, alginates, stearates, starches, rubbers and cellulose ethers, as described in DE-C-4100920, be brought into a state in which it is stable against digestion by animal stomachs, in particular the stomach of ruminants.

To set a desired concentration of L-lysine in the product, depending on requirements, during the process L-lysine can be added in the form of a concentrate, or if appropriate a substantially pure substance, or salt thereof, in liquid or solid form. These can be added individually or as mixtures to the fermentation broth obtained or concentrated fermentation broth, or else during the drying or granulation process.

The invention further relates to a process for producing a solid L-lysine-containing product, as described in its basics in US 20050220933, and which comprises workup of the fermentation broth obtained using the bacteria produced according to the invention:
a) filtration of the fermentation broth, preferably using a membrane filter, such that a biomass-containing sludge and a filtrate is obtained,
b) concentration of the filtrate, preferably in such a manner that a solids content of 48 to 52% by weight is obtained,
c) granulation of the concentrate obtained in step b), preferably at a temperature of 50° C. to 62° C., and
d) coating the granules obtained in c) with one or more of the coating agent(s).

For coating in step d), use is preferably made of coating agents which are selected from the group consisting of
d1) the biomass obtained in step a),
d2) a L-lysine-containing compound, preferably selected from the group L-lysine hydrochloride or L-lysine sulfate,
d3) an essentially L-lysine-free material having an L-lysine content <1% by weight, preferably <0.5% by weight, preferably selected from the group starch, carageenan, agar, silicas, silicates, coarse meals, brans and flours, and
d4) a water-repelling substance, preferably selected from the group oils, polyethylene glycols and liquid paraffins.

In the production of the L-lysine-containing products, the ratio of the ions is preferably set in such a manner that the equivalent ion ratio corresponding to the formula below

2x[SO₄²⁻]+[Cl⁻]−[NH₄⁺]−[Na⁺]−[K⁺]−2x[Mg²⁺]−2x[Ca²⁺]/[L-Lys]

gives 0.68 to 0.95 preferably 0.68 to 0.90, as described by Kushiki et al. in US 20030152633 (the molar concentrations are to be reported in “[ ]”.)

The L-lysine-containing solid product produced in this manner on a fermentation broth base has an L-lysine content (as lysine base) of 10% by weight to 70% by weight, or 20% by weight to 70% by weight, preferably 30% by weight to 70% by weight, and very particularly preferably 40% by weight to 70% by weight, based on the dry mass of the product. Maximum contents of lysine base of 71% by weight, 72% by weight, 73% by weight are likewise possible.

The water content of the solid product is up to 5% by weight, preferably up to 4% by weight, and particularly preferably less than 3% by weight.

The invention therefore also relates to an L-lysine-containing feed additive on a fermentation broth base, which has the following features
a) an L-lysine content (as base) of at least 10% by weight to a maximum of 73% by weight, based on the total amount of the additive,
b) a water content of at most 5% by weight, and
c) a biomass content corresponding to at least 0.1% of the biomass present in the fermentation broth, if appropriate, inactivated biomass consisting of the bacteria formed in the process according to the invention.

1-27. (canceled)

28. A process for producing L-lysine or L-lysine-containing feed additives by fermentation, comprising:
a) expressing a polynucleotide coding for a polypeptide having LL-diaminopimelate aminotransferase activity in an L-lysine excreting bacterium; and
b) fermenting the resultant bacterium in a medium under suitable conditions and allowing the resultant L-lysine excreted by the bacterium to accumulate in the fermentation broth.

29. The process of claim 28, wherein the polynucleotide coding for a polypeptide having LL-diaminopimelate aminotransferase activity is a polynucleotide from Arabidopsis thaliana, Synechococcus sp., Oryza sativa or Vitis vinifera.

30. The process of claim 28, wherein the bacterium is a bacterium selected from the group coryneform bacteria, the family Enterobacteriaceae and the genus Bacillus.

31. The process of claim 30, wherein the coryneform bacterium is a bacterium of the genus Corynebacterium.

32. The process of claim 31, wherein the bacterium of the genus Corynebacterium is a bacterium selected from the group Corynebacterium glutamicum, Corynebacterium efficiens, Corynebacterium callunae, Corynebacterium thermoaminogenes and Corynebacterium ammoniagenes.

33. The process of claim 32, wherein the bacterium is Corynebacterium glutamicum.

34. The process of claim 30, wherein the bacterium of the family Enterobacteriaceae is a bacterium of the genus Escherichia.

35. The process of claim 34, wherein the bacterium of the genus Escherichia is Escherichia coli.

36. The process of claim 30, characterized in that the bacterium of the genus Bacillus is Bacillus subtilis or Bacillus methanolicus.

37. The process of claim 28, wherein said bacterium excretes at least 0.25 g/l L lysine in maximally 120 hours.

38. The process of claim 28, wherein bacterium possesses a gene coding for a feedback resistant aspartate kinase.

39. The process of claim 38, wherein the polypeptide has an amino acid sequence which is at least 70% identical to the amino acid sequences selected from the group: SEQ ID NO: 2 SEQ ID NO: 4, SEQ ID NO: 6, SEQ ID NO: 8, and SEQ ID NO: 10.

40. The process of claim 39, wherein the polypeptide has an amino acid sequence which is identical to an amino acid sequence selected from the group: SEQ ID NO: 2 SEQ ID NO: 4, SEQ ID NO: 6, SEQ ID NO: 8, and SEQ ID NO: 10.

41. The process of claim 28, wherein the bacterium is fermented in a fed-batch process.

42. The process of claim 28, wherein the bacterium is fed in a repeated fed-batch process.

43. The process of claim 28, wherein the bacterium is fermented in a continuous process.

44. The process of claim 28, wherein the L-lysine excreted by the bacterium is collected.

45. The process of claim 28, wherein the L-lysine excreted by the bacterium is obtained together with components of the fermentation broth.

46. The process of claim 28, wherein the L-lysine excreted by the bacterium is isolated and purified.

47. An L-lysine-excreting bacterium which has been transformed by a polynucleotide coding for a polypeptide having LL-diaminopimelate aminotransferase activity and, compared with a non-transformed bacterium, exhibits increased excretion of L-lysine.

48. The L-lysine-excreting bacterium of claim 47, wherein the polypeptide having LL-diaminopimelate aminotransferase activity is a polynucleotide of Arabidopsis thaliana, Synechococcus sp., Oryza sativa or Vitis vinifera.

49. The L-lysine-excreting bacterium of claim 47, wherein the bacterium is a coryneform bacterium.

50. The L-lysine-excreting bacterium of claim 49, wherein the bacterium is a coryneform bacterium of the genus Corynebacterium.

51. The L-lysine-excreting bacterium of claim 50, wherein the bacterium of the genus Corynebacterium is Corynebacterium glutamicum.

52. The L-lysine-excreting bacterium of claim 47, wherein the bacterium is a bacterium of the family Enterobacteriaceae.

53. The L-lysine-excreting bacterium of claim 52, wherein the bacterium of the family Enterobacteriaceae is of the genus Escherichia.

54. The L-lysine-excreting bacterium of claim 53, wherein the bacterium of the genus Escherichia is Escherichia coli.


SEQUENCE LISTING (12 sequences)

SEQ ID NO: 1 - DNA, 1386 bp, Arabidopsis thaliana
  CDS (1)..(1383)
atgtcgtcgacccatcagttagtttcttcgatgatctcttcttcctcatccactttcttagccccttcaaattttaatctcagaactcgaaatgcttgcttacccatggcaaaacgggtcaatacttgcaaatgtgttgctacgccgcaagagaagatcgagtataagaccaaagtgtcacggaattcaaacatgtccaaacttcaggctggatacctattcccggagattgcaagaagaaggtctgcacacttgttgaaatatccagatgcacaagttataagtcttggaataggcgacacaactgagccaattcctgaagtgatcacttctgctatggcaaagaaagctcatgagttgtcaacaatagagggatatagtggttatggtgctgaacaaggtgcaaagccactgagagctgctattgcgaaaacattctacggtggccttggcataggggatgatgacgtttttgtttctgatggagctaaatgtgatatctcacgtctccaggttatgtttggttccaatgttacaattgctgttcaggatccttcatatccggcttatgtggactccagtgttattatgggtcagactgggcaatttaacactgatgtgcaaaagtatggaaacatcgagtacatgagatgcactccagagaatggcttctttcccgacttgtccaccgttggcaggacagatataattttcttctgttccccaaataaccctacgggtgctgctgccacgagagagcaactaacgcagttagttgagtttgcaaagaagaacggttctataatagtgtatgattccgcctatgcaatgtacatgtctgatgataacccacgatccatcttcgaaatccctggagcagaggaggtcgctatggagacagcttcgttcagcaaatatgctggtttcactggagttcgacttggttggactgtcatcccgaaaaagctactctattcagacggtttccctgttgccaaagacttcaatcggattatctgcacttgtttcaatggtgcatctaatatctctcaagctggtgctcttgcttgccttacacccgaaggacttgaggcaatgcataaggtgattggattctataaagaaaacacaaacataatcattgacacattcacatctctcgggtatgatgtatatggaggaaagaatgcgccttacgtatgggttcacttcccgaaccaaagctcatgggatgtgtttgctgagattctggagaagactcatgtggttacaactccaggaagtgggtttggaccagggggtgaagggttcgttcgtgtcagtgcctttggtcacagagagaacatcttagaggcatgtcgaagattcaagcagctttacaaatga

SEQ ID NO: 2 - protein, 461 aa, Arabidopsis thaliana [sequence omitted]

SEQ ID NO: 3 - DNA, 1239 bp, Synechococcus
  CDS (1)..(1236)
atggccagtatcaacgacaactatctcaaactgaaagccggttacctgtttcccgaaattgctcggcgggtaaatgctttcaccactgccaatcctaacgcccaggtgatcaaacttggtattggagatgtaacggaacccctgccccttgcttgtcgccaggccatggccaaagccatcgacgacatgggcgatcgccaaaccttcaaaggatacggcccggaacagggttacgcttggttgcgggaaaaaattgcccagcacgatttccaagctaggggctgtgaggttaacgccgaagaaattttcatctccgacggttctaaatgtgacaccggcaatatcctcgatattttcggcaaagataacaccatcgctgtcactgaccctgtttatcccgtttatgtggacaccaatgtgatggcgggccataccggcgatgccaatgaaaaaggggaatacggtggtttagtctatctccccatttcggccgaaaatgactttgtcgcggccataccaagcaaaaaagtagatttaatctatctctgttttcccaacaaccccaccggagccactgccactaaggcctatttgaaacagtgggtagattatgccctggcccacggctcgatcatctttttcgatgcggcctacgaggcatttatcactgaccccactttgccccattccatctatgaaattgaaggagcgagggattgcgccattgagtttcgctctttttctaaaaatgctggtttcaccggtacccgttgtgcgttaaccgttgtgcccaaaaccttaacggctaaagccgccgacggcagtgacgtggagctatggaaactctggaaccgccgccaatccaccaaatttaatggcgtttcctacattatccagcgaggggcagaggccgtctattcccccgaaggccaagcccaagtacaggaactgattgctttttatttggaaaatgctcgcattattcgggagaaattagccgccgctggtttgcaggtttatggtggcattaacgctccctatgtgtgggtcaaaaccccccatggcctgagcagttgggactttttcgataagttattacacacagttaatgtggtgggcactccaggctctggctttggcgcggcgggggaaggctatttccgcatttcggctttcaatagtcgggccaatgtggaggaagcgatggaacgaatcacttccaccctcaaattgggttag

SEQ ID NO: 4 - protein, 412 aa, Synechococcus
MASINDNYLKLKAGYLFPEIARRVNAFTTANPNAQVIKLGIGDVTEPLPLACRQAMAKAIDDMGDRQTFKGYGPEQGYAWLREKIAQHDFQARGCEVNAEEIFISDGSKCDTGNILDIFGKDNTIAVTDPVYPVYVDTNVMAGHTGDANEKGEYGGLVYLPISAENDFVAAIPSKKVDLIYLCFPNNPTGATATKAYLKQWVDYALAHGSIIFFDAAYEAFITDPTLPHSIYEIEGARDCAIEFRSFSKNAGFTGTRCALTVVPKTLTAKAADGSDVELWKLWNRRQSTKFNGVSYIIQRGAEAVYSPEGQAQVQELIAFYLENARIIREKLAAAGLQVYGGINAPYVWVKTPHGLSSWDFFDKLLHTVNVVGTPGSGFGAAGEGYFRISAFNSRANVEEAMERITSTLKLG

SEQ ID NO: 5 - DNA, 1395 bp, Oryza sativa
  CDS (1)..(1392)
atggccgcctcccccgccgccggggccgcggccgccaccgtctcgtcgttcgtctcgccgtcgtccttctcctccgtcaaggcatcgaagcccgaccgcctccgtccggcgaggagggcggcggccgtcaacgtccgatgcgtcagcagccctccggccaccgaaacttctttcaagacgaaagtaccacgaaatgccaatatggccaagctgcaagctggctatctgtttcctgagattgccaggagaagggcagctcacctgctgaagtttcctgatgcgaagattataagccttgggatcggtgacactacagagcccattccagacgtcataactaatgccatggcaaagagagcacatgctttatccacagttgatggttatagtggttatggagctgaacagggtgaaaagaaactaagggcagctattgctgcaacctactatgcggatcttggtattgaagaaacagatatattcgtctctgatggtgcaaaatgtgacatttctcgtctgcaggtcctttttggatctaatgtgaagattgcagttcaagatccatcatatcctgcctatgttgattcaagtgttatcatggggcaaactggtttatatcaggaggatgttcagaagtatggaaatattgagtacatgaaatgcagtcctgaaaatgggtttttccctgatctgtcaagtgttccacgaacagatataatcttcttttgttcacccaacaatcctactggtgctgctgcatctcgggaccaattaaccaaattagtcaaatttgcaaaggacaatggatccattatagtttatgattctgcttatgccatgtacatatcagatgacagcccaaaatctatctttgaaattcctggagcaaaggaggttgccattgaaacagcatctttctccaaatatgctggattcactggtgtgcgtttgggttggactgtggttcctaaggagctccttttctcagatgggcatcctgttgctaaagatttcaaccgcatagtctgcacttgcttcaatggtgcatcaaacatttctcaagccggtggtttaggttgtctatctccagagggtctaaaggccatgagtgacgttgtcggcttctacaaggaaaatactaaaattattgttgacacatttacatcacttggattcaacgtctacggtgccaagaatgctccatatgtgtgggtgcacttccctggccgtaattcttgggatgtcttcgccgaaattcttgagaaggcacatgtggttactactcctggtagcggatttgggcccggtggtgaaggcttcgtaagggtcagtgcatttgggcacagagagaacattattgaagctgcgagaagactgaagcagctgtacaaatga

SEQ ID NO: 6 - protein, 464 aa, Oryza sativa
MAASPAAGAAAATVSSFVSPSSFSSVKASKPDRLRPARRAAAVNVRCVSSPPATETSFKTKVPRNANMAKLQAGYLFPEIARRRAAHLLKFPDAKIISLGIGDTTEPIPDVITNAMAKRAHALSTVDGYSGYGAEQGEKKLRAAIAATYYADLGIEETDIFVSDGAKCDISRLQVLFGSNVKIAVQDPSYPAYVDSSVIMGQTGLYQEDVQKYGNIEYMKCSPENGFFPDLSSVPRTDIIFFCSPNNPTGAAASRDQLTKLVKFAKDNGSIIVYDSAYAMYISDDSPKSIFEIPGAKEVAIETASFSKYAGFTGVRLGWTVVPKELLFSDGHPVAKDFNRIVCTCFNGASNISQAGGLGCLSPEGLKAMSDVVGFYKENTKIIVDTFTSLGFNVYGAKNAPYVWVHFPGRNSWDVFAEILEKAHVVTTPGSGFGPGGEGFVRVSAFGHRENIIEAARRLKQLYK

SEQ ID NO: 7 - DNA, 1263 bp, Corynebacterium glutamicum
  CDS (1)..(1263): lysC-Wildtyp-Gen
gtggccctggtcgtacagaaatatggcggttcctcgcttgagagtgcggaacgcattagaaacgtcgctgaacggatcgttgccaccaagaaggctggaaatgatgtcgtggttgtctgctccgcaatgggagacaccacggatgaacttctagaacttgcagcggcagtgaatcccgttccgccagctcgtgaaatggatatgctcctgactgctggtgagcgtatttctaacgctctcgtcgccatggctattgagtcccttggcgcagaagcccaatctttcacgggctctcaggctggtgtgctcaccaccgagcgccacggaaacgcacgcattgttgatgtcactccaggtcgtgtgcgtgaagcactcgatgagggcaagatctgcattgttgctggtttccagggtgttaataaagaaacccgcgatgtcaccacgttgggtcgtggtggttctgacaccactgcagttgcgttggcagctgctttgaacgctgatgtgtgtgagatttactcggacgttgacggtgtgtataccgctgacccgcgcatcgttcctaatgcacagaagctggaaaagctcagcttcgaagaaatgctggaacttgctgctgttggctccaagattttggtgctgcgcagtgttgaatacgctcgtgcattcaatgtgccacttcgcgtacgctcgtcttatagtaatgatcccggcactttgattgccggctctatggaggatattcctgtggaagaagcagtccttaccggtgtcgcaaccgacaagtccgaagccaaagtaaccgttctgggtatttccgataagccaggcgaggctgcgaaggttttccgtgcgttggctgatgcagaaatcaacattgacatggttctgcagaacgtctcttctgtagaagacggcaccaccgacatcaccttcacctgccctcgttccgacggccgccgcgcgatggagatcttgaagaagcttcaggttcagggcaactggaccaatgtgctttacgacgaccaggtcggcaaagtctccctcgtgggtgctggcatgaagtctcacccaggtgttaccgcagagttcatggaagctctgcgcgatgtcaacgtgaacatcgaattgatttccacctctgagattcgtatttccgtgctgatccgtgaagatgatctggatgctgctgcacgtgcattgcatgagcagttccagctgggcggcgaagacgaagccgtcgtttatgcaggcaccggacgc

SEQ ID NO: 8 - protein, 421 aa, Corynebacterium glutamicum
VALVVQKYGGSSLESAERIRNVAERIVATKKAGNDVVVVCSAMGDTTDELLELAAAVNPVPPAREMDMLLTAGERISNALVAMAIESLGAEAQSFTGSQAGVLTTERHGNARIVDVTPGRVREALDEGKICIVAGFQGVNKETRDVTTLGRGGSDTTAVALAAALNADVCEIYSDVDGVYTADPRIVPNAQKLEKLSFEEMLELAAVGSKILVLRSVEYARAFNVPLRVRSSYSNDPGTLIAGSMEDIPVEEAVLTGVATDKSEAKVTVLGISDKPGEAAKVFRALADAEINIDMVLQNVSSVEDGTTDITFTCPRSDGRRAMEILKKLQVQGNWTNVLYDDQVGKVSLVGAGMKSHPGVTAEFMEALRDVNVNIELISTSEIRISVLIREDDLDAAARALHEQFQLGGEDEAVVYAGTGR

SEQ ID NO: 9 - DNA, 1380 bp, Vitis vinifera
  CDS (1)..(1377)
atgtactctctcatgtcttcgccttccatctcttcatcttcctcctcctttttaggccaaacccacttcgattccagaaacccaaatgcttgccttcccacaaaagatagttggatatgtaaatgtgtggcaacaccttccacagagacgactgctcacacgacaaaggtctctcgcaatgcaaatatggccaaacttcaagctggttatttgtttccagagatagccagaagaaggtcagcacacatgctaaagtaccctgatgcacaagtgataagccttggaattggtgacactactgagcccattccagaagttataacttctggtatggcaaagaaagcacatgcattgtcaacactagagggttacagtggttatggagctgaacaaggtgaaaaacaattgagagctgcaattgcttcgaccttttatggtgacctcagcatagaggaaagtgatatatttgtctctgatggtgcaaaatctgacatatctcgccttcaggtcatgtttgggtctaatgttacaatggcagtgcaagacccatcatacccggcttatgtagacttaagtgttatcttgggccagactgggcagtttcagaaggatgttgagaagtatggaaacatcgaatacatgaagtgtaatccagagaatggtttctttcctgatttatctactgtttcgcgaaccgatatcatttttttctgttcaccatacaatcccactggtaatgctgcaacaagggagcaactgacccgacttgtacagtttgccaaggacaatggatcaattctagtgtatgattcgggatatgccatgtatatctcggatgacagcccaaggtccatctttgaaattcctggagccaaagaggttgcaattgaggtatcatcattttccaaatatgctgggttcactggagtccgtcttggttggactgtggttccaaaggagcttctatattcagatggatttcctgttgccaaggactttaaccgcattgaatgtacaaccttcaacgctgcatccaacatttctcaagctagtggtctggcttgtctttcaccagaaggcctagaggctatgcataaactggtaggtttctacaaagaaaatactaatataatcatggagacattcacttctcttggttttagcgtgtacggaggtaagaatgcaccatatgtttgggttcatttccctggtcaaagctcatgggacgtgttcagcgagatacttgagaagacccatgtggtaacaacacctggcagtggtttcggaccagctggtgatggtttcattagggtttgtgcttttagtcacagaggcaatgttctagaggcctgcaaaagattcaagcggttatacaagtga

SEQ ID NO: 10 - protein, 459 aa, Vitis vinifera
MYSLMSSPSISSSSSSFLGQTHFDSRNPNACLPTKDSWICKCVATPSTETTAHTTKVSRNANMAKLQAGYLFPEIARRRSAHMLKYPDAQVISLGIGDTTEPIPEVITSGMAKKAHALSTLEGYSGYGAEQGEKQLRAAIASTFYGDLSIEESDIFVSDGAKSDISRLQVMFGSNVTMAVQDPSYPAYVDLSVILGQTGQFQKDVEKYGNIEYMKCNPENGFFPDLSTVSRTDIIFFCSPYNPTGNAATREQLTRLVQFAKDNGSILVYDSGYAMYISDDSPRSIFEIPGAKEVAIEVSSFSKYAGFTGVRLGWTVVPKELLYSDGFPVAKDFNRIECTTFNAASNISQASGLACLSPEGLEAMHKLVGFYKENTNIIMETFTSLGFSVYGGKNAPYVWVHFPGQSSWDVFSEILEKTHVVTTPGSGFGPAGDGFIRVCAFSHRGNVLEACKRFKRLYK

SEQ ID NO: 11 - DNA, 959 bp, Corynebacterium glutamicum
  promoter (664)..(822)
  misc_feature (834)..(836): ATG start codon of gltA cds
tccgtcgacaatagcctgaatctgttctggtcgaaccttggaaggtccgcagccgaaacggccgtcgccagggatgaactcagagggcagggtggggaagtcggtcatgtcttcgggcaactttctgcgcttggaagtaaaagggccagggatcgttaacgatctgacccaacaactataaccctgaagctgtcagttcctagcaccctagattcttcacgcagtctcccaaacgatgaaaaacgcccaaaactggcgacaccgaactattgaaaacgcggggattagttgaccagccaccaatttgggggtagctcaaagttttgcaaagttttcaatttctaggttgttaatatcccctgaggttgcgttatagggtggcgaattgcatggggaaagctactcggcacccatccttgtcgcgtgcatcacaaactttgctaaactgtgcaccagtccacttattgtgggatttttaatgccttaaaggccagcattttcaccctctagcggggttgaatgctggccttgagggtgcagaactaaatagcagcacatcggcacaattgatctgagttctattggcgtgaccgtggctactgattacggtggctgtgggtggtcgggaatgatgtaaccaacgtgattgtgggggaattggctctcacttcggatatggctaaaccgcatttatcggtatagcgtgttaaccggaccagattgggaaagaaatgtgtcgagtaacaaaaactgacatgcgcttggcgcatcccagttggtaagaataaacgggactacttccgtaatccggaagagtttttttccgaacaaatatgtttgaaagggatatcgtggctactgataacaacaaggctgtcctgcactaccccggtggcgagttcgaaatggacatcatcgaggcttctgagggtaacaacggtgttgtcctgggcaagatg

SEQ ID NO: 12 - protein, 42 aa, Corynebacterium glutamicum
MFERDIVATDNNKAVLHYPGGEFEMDIIEASEGNNGVVLGKM